SEQUENCE LISTING (2 sequences)

SEQ ID NO: 1 - DNA, 20 bp, Homo sapiens
gtctctttcactcagccaac

SEQ ID NO: 2 - DNA, 20 bp, Homo sapiens
atcaggatttctcccctcag

(19) United States
(12) Patent Application Publication
(10) Pub. No.: US 2014/0170162 A1
(43) Pub. Date: Jun. 19, 2014

(54) PRESERVATION OF THE NEUROMUSCULAR JUNCTION (NMJ) AFTER TRAUMATIC NERVE INJURY

(71) Applicant: The Regents of the University of California, Oakland, CA (US)

(72) Inventor: Ranjan Gupta, Irvine, CA (US)

(73) Assignee: THE REGENTS OF THE UNIVERSITY OF CALIFORNIA, Oakland, CA (US)

(21) Appl. No.: 14/133,414

(22) Filed: Dec. 18, 2013

Related U.S. Application Data
(60) Provisional application No. 61/738,912, filed on Dec. 18, 2012.

(51) Int. Cl. C12N 15/113; A61K 39/395; A61K 31/65; A61K 31/165; A61K 31/496; A61K 31/4709; A61K 31/35; A61K 31/506; A61K 31/513; A61K 31/365; A61K 38/17; A61K 31/519

(52) U.S. Cl. CPC C12N 15/1138; A61K 38/1709; C12N 15/1137; A61K 39/3955; A61K 31/65; A61K 31/165; A61K 31/519; A61K 31/4709; A61K 31/35; A61K 31/506; A61K 31/513; A61K 31/365; A61K 31/496
USPC 424/158.1; 514/18.1; 514/44 A; 514/154; 514/575; 514/260.1; 514/313; 514/456; 514/252.14; 514/274; 514/449; 514/253.06; 514/252.18

(57) ABSTRACT

The invention relates to treatment and/or prevention of nerve injury. In one embodiment, the present invention provides a method of preserving the neuromuscular junction (NMJ) in an individual by administering a therapeutically effective dosage of a composition comprising an inhibitor of Wnt3a, and an inhibitor of MMP3 to the individual. In another embodiment, the present invention provides a method of stabilizing NMJ after nerve injury by inhibiting the WNT and beta-catenin signaling pathway and preserving agrin.

### CROSS-REFERENCE TO RELATED APPLICATIONS

The present application claims the benefit of priority under 35 U.S.C. §119(e) of provisional application Ser. No. 61/738,912, filed Dec. 18, 2012, the contents of which are hereby incorporated by reference.

### FIELD OF USE

This invention relates generally to the field of medicine and, in particular, to methods and compositions for treating nerve injury.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Although the peripheral nervous system has the capacity for regeneration following injury, functional recovery after neural repair in adult humans remains limited. Despite surgical repair, there often still remains a poor outcome where the patient experiences only limited functional motor recovery. Some of the issues that may be associated with peripheral nerve regeneration include a lack of good scaffolding for regeneration, glial scar formation, poor peripheral support, and imprecise connections resulting in lack of coordination. In response, one strategy would be to focus on the preservation of the neuromuscular junction. The neuromuscular junction contains three cellular components, namely the terminal branch of the motor axon, the terminal schwann cell or perisynaptic Schwann cell, and muscle fiber with acetylcholine reeptors (AChRs). Degradation of the motor endplate could render the target organ nonviable for the regenerating nerve despite reaching the target. There is a need in the art to develop novel and effective treatments for nerve injury beyond the more commonly used surgical procedures.

### SUMMARY OF THE INVENTION

Various embodiments include a method of treating nerve injury in an individual, comprising providing a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway, and administering a therapeutically effective dosage of the composition to the individual. In another embodiment, the composition is administered in conjunction with surgical treatment. In another embodiment, the individual is a human. In another embodiment, the inhibitor of the MMP3 signaling pathway is an inhibitor of MMP3. In another embodiment, the inhibitor of the WNT signaling pathway is an inhibitor of Wnt3a. In another embodiment, the nerve injury is treated by preserving the neuromuscular junction (NMJ). In another embodiment, administering the composition prevents degradation of the motor end plate after prolonged denervation. In another embodiment, the composition is administered prior to nerve injury surgery. In another embodiment, the composition is administered post nerve injury surgery. In another embodiment, the composition is administered intravenously. In another embodiment, the inhibitor of the MMP3 signaling pathway is selected from the following: minocycline, MMP Inhibitor II, MMP Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618. In another embodiment, the inhibitor of the MMP3 signaling pathway is an MMP3 siRNA molecule. In another embodiment, the inhibitor of the WNT signaling pathway is an Wnt3a siRNA molecule. In another embodiment, the inhibitor of the WNT signaling pathway is an inhibitor of the armadillo protein β-catenin. In another embodiment, the inhibitor of the WNT signaling pathway is an inhibitor of one or more of the following: beta-catenin destruction complex, WNT/Beta-catenin signalsome, cadherin junctions, and hypoxi sensing system Hif-1alpha (hypoxia induced factor 1beta). In another embodiment, the inhibitor of the WNT signaling pathway is one or more of the following: XAV939, IWR1, IWP-1, IWP-2, JW74, JW55, okadaic acid, tautomycein, 2-[4-(4-fluoro-phenyl)piperazin-1-yl]-6-methylpyrimidin-4(3H)-one, niclosamide, cambinol, sulindac, filipin, bosutinib, imatinib, ethacrynic acid, PKF118-744, BC21, and Rp-8-Br-cAMP.

Other embodiments include a composition comprising a therapeutically effective dosage of a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway, and a pharmaceutically acceptable carrier. In another embodiment, the inhibitor of the MMP3 signaling pathway is an inhibitor of MMP3. In another embodiment, the inhibitor of MMP3 is an MMP3 antibody. In another embodiment, the inhibitor of MMP3 is selected from the following: minocycline, MMP Inhibitor II, MMP Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618. In another embodiment, the inhibitor of the WNT signaling pathway is an inhibitor of Wnt3a. In another embodiment, the inhibitor of Wnt3a is an Wnt3a antibody. In another embodiment, the inhibitor of MMP3 signaling pathway is selected from the following: XAV939, IWR1, IWP-1, IWP-2, JW74, JW55, okadaic acid, tautomycein, 2-[4-(4-fluoro-phenyl)piperazin-1-yl]-6-methylpyrimidin-4(3H)-one, niclosamide, cambinol, sulindac, filipin, bosutinib, imatinib, ethacrynic acid, PKF118-744, BC21, and Rp-8-Br-cAMP.

Other embodiments include a method of preventing nerve injury in an individual, comprising providing a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway, and administering a therapeutically effective dosage of the composition to the individual prior to nerve injury. In another embodiment, the composition is administered intravenously.

Various other embodiments include a methods of preserving the motor end plate after nerve injury in a subject, comprising providing a composition comprising MMP3 pathway specific siRNA, WNT pathway specific siRNA, and beta-catenin pathway specific siRNA; and transfecting one or more cells of the subject with the composition. In another embodiment, the composition comprises SEQ. ID. NO.: 1 and SEQ. ID. NO.: 2. In another embodiment, the subject is a human. In another embodiment, the subject is a rodent.

Other features and advantages of the invention will become apparent from the following detailed description, taken in conjunction with the accompanying drawings, which illustrate, by way of example, various embodiments of the invention.

### BRIEF DESCRIPTION OF THE FIGURES

FIG. 1 depicts, in accordance with an embodiment herein, creation of long-term denervation model for tibialis anterior muscle (TA). (A) The sciatic nerve (SN) separates into sensory and motor branches upon exiting the sciatic notch. The 2 motor branches, the common peroneal (C) and the tibial nerve (Tib) branch, are shown. Denervation of the TA muscle was accomplished by transection of the common peroneal nerve and suturing the proximal (Cp) and distal (Cd) segments into the adjacent musculature. (B) The TA muscle was innervated following 2 months denervation by nerve transfer of the tibial nerve (Tibp) to the distal common peroneal stump (Cd). The distal segment of the tibial nerve (Tibd) was then ablated to prevent aberrant regeneration into the stump.

FIG. 2 depicts, in accordance with an embodiment herein, agrin (Agr) and muscle-specific kinase (MuSK) remain at the motor endplate during denervation in MMP3 null mice. (A-D, I-J) Agrin immunostaining for wild-type (WT) and matrix metalloproteinase 3 (MMP3) null mice at baseline, 1 month, and 2 months postdenervation. Note colocalization of agrin with Schwann cell processes and absence of agrin immunoreactivity in WT mice in the acetylcholine receptor region. Significant upregulation of agrin was seen throughout the muscle substance following injury (asterisk). (E-H) MuSK immunostaining for WT and MMP3 null mice at baseline and 1 month postdenervation. (K) Western blot for neural agrin following immunoprecipitation with LRP4. At 1 month of denervation, minimal neural agrin is seen in WT specimens, whereas substantial amounts of neural agrin are present in denervated knockouts (KO). Agrin band measures approximately 95 kD. LRP4 (216 kD) is shown as internal control. (L) Bands representing phosphorylated MuSK (PY) at 7, 14, and 30 days of denervation in WT and MMP3 knockout mice. MuSK phosphorylation decreases gradually in WT mice, whereas the amount of MuSK phosphorylation remains constant in MMP3 knockout mice. Total MuSK bands representing loading control are shown along the bottom row. Band measures approximately 110 kD. (M, N) Knockout mice were seen to have a higher percentage of phosphorylated MuSK (74.9%) compared to WT mice (36.6%) 2 months following denervation. BTX 5 a-AQ7 bungarotoxin; IgG 5 immunoglobulin G. Antibody to S100 protein was used to identify perisynaptic Schwann cells. Scale bars 5 1 lm.

FIG. 3 depicts, in accordance with an embodiment herein, (A, B) Acetylcholine receptor (AChR) clustering secondary to agrin is blocked via matrix metalloproteinase 3 (MMP3) in vitro. Images (original magnification, 340) of AChRs in C212 myotubes show clustering in the presence of agrin alone (A) and no clustering with agrin and MMP3 (B). (C) Graphical representation of number of AChR clusters per field seen. There was a significant difference in the number of AChR clusters seen in the presence of agrin versus agrin and MMP3. (D) Silver staining for commercial agrin incubated with and without MMP3 for 24 versus 72 hours. Agrin measures 90 kDa and the cleavage product via MMP3 is 60 kDa. (E) Western blot for agrin incubated with and without MMP3 for 24 hours. **p<0.01. Graphical bars represent standard error of the mean. BTX 5 a-bungarotoxin.

FIG. 4 depicts, in accordance with an embodiment herein, characterization of wild-type (WT) and matrix metalloproteinase 3 (MMP3) knockout (KO) species. (A-F) Immunohistochemistry for MMP3 protein in both WT and KO animals demonstrates absence of signal at the motor endplate in KO mice. (G) Western blot for MMP3 protein confirms inability to detect the enzyme in knockout animals. Band size was measured at approximately 50 kDa. Glyceraldehyde-3-phosphate dehydrogenase (GAPDH) is shown as internal control. (H) Total mass of 6-week-old WT and MMP3 null mice are approximately equal. (I) Sciatic function index (SFI) measurements for WT and MMP3 animals obtained prior to denervation injury. WT value was set at 210. Calculation of SFI in MMP3 KO animals used WT animals as reference. One sample Student t test was used to analyze for statistical significance. Standard error of the mean is noted on all graphs. p 5 0.480. Original magnification, 3100. Scale bar 5 1 lm. BTX 5 a-bungarotoxin.

FIG. 5 depicts, in accordance with an embodiment herein, matrix metalloproteinase 3 (MMP3) null mice resist derangement in acetylcholine receptor (AChR) area and morphology after denervation. (A-H) Images (original magnification, 340; scale bars 5 15 l) of the AChRs are shown for wild-type (WT) and MMP3 knockout (KO) mice at baseline and 7, 14, and 30 days after denervation. (I, J) Receptor area and pixel density decreased to a lesser degree in MMP3 null mice than in WT animals up to 30 days following transection. Forty-five receptors were characterized from each muscle. **p<0.01. (K-M) Representation of the morphology of AChRs encountered in muscle preparations. (N, O) A shift in the receptor morphology toward plaquelike profiles was much more pronounced in WT specimens by the 30-day time point. In contrast, MMP3 null animals contained a larger percentage of intermediate receptors at 30 days of denervation. Photographs for the pretzel, intermediate, and plaque phenotypes are shown. **p<0.01. (P, Q) The alpha subunit decreases more substantially in WT mice than in MMP3 null mice in response to denervation by the 30-day time AQ8 point. Visualized band was identified at approximately 55 kDa. **p value<0.01. Glyceraldehyde-3-phosphate dehydrogenase (GAPDH) is shown as internal control. Graphical bars indicate standard error of the mean.

FIG. 6 depicts, in accordance with an embodiment herein, analysis of motor endplates at 2-month denervation. (A, B) The acetylcholine receptor band remained intact at 1-month denervation in wild-type and matrix metalloproteinase 3 (MMP3) null mice but was disbanded by 2 months in wild-type mice. Original magnification, 310; scale bar 5 160 l. (C, D) Images (original magnification, 340; scale bar 5 40 l) confirmed dispersion in the wild-type but not the MMP3 null mouse. (E) Band intensity measurements for 2-month muscles showing significant loss of optical density in wild-type specimens. (F) Number of endplate counts at 2 months of denervation showing significant decrease in number of endplates in wild-type animals. *p<0.05; **p value<0.01. Original magnification, 3100. BTX 5 a-bungarotoxin; KO 5 knockout. Graphical bars represent standard error of mean.

FIG. 7 depicts, in accordance with an embodiment herein, wild-type and matrix metalloproteinase 3 (MMP3) knockout (KO) mice undergo similar denervation-related processes. (A-D) The nerve terminal was seen to retract from the motor endplate in both wild-type and MMP3 null mice. By the 30-day time point, all neural elements had vacated their targets. Normal and 30-day denervated receptor profiles are shown (original magnification, 3100). (E-H) Likewise, Schwann cells failed to express 5100 at the motor endplate at 30 days of denervation in both wild-type and MMP3 null mice. (I-L) Muscle cross-sectional area decreased equally in both wild-type and MMP3 soleus muscles following denervation at 7, 14, and 30 days postinjury. Uninjured and 1-month denervated images are shown. (M) Graphical representation of cross-sectional analysis. No difference in the rate of muscle atrophy between wild-type and MMP3 knockout mice was observed. A significant amount of atrophy was seen during the first 2 weeks of denervation in both animal groups. Images for muscle cross sections are displayed (original magnification, 320). BTX 5 a-bungarotoxin; NF 5 neurofilament; Syn 5 synaptophysin. Antibody to 5100 protein was used to identify perisynaptic Schwann cells. Bars on graphs indicate standard error of the mean. Scale bars 5 1 lm (A-H) and 30 lm (I-L).

FIG. 8 depicts, in accordance with an embodiment herein, muscles from denervated matrix metalloproteinase 3 (MMP3) knockout mice demonstrated higher contractile force in response to ex vivo acetylcholine stimulation than wild-type counterparts. (A) Muscle length was approximately equal among all 4 groups tested: 0.698 6 0.0699 cm (wild-type normal), 0.631 6 0.116 cm (wild-type denervated), 0.658 6 0.0596 cm (knockout normal), and 0.634 6 0.145 cm (knockout denervated). (B) Muscle mass was equal in wild-type and MMP3 knockouts under similar injury conditions: uninjured (15.5 6 0.936 g vs 14.6 6 3.61 g) and 1-month denervated specimens (7.34 6 1.04 g vs 7.68 6 1.84 g). **p<0.001. (C) Force measurements for wild-type and MMP3 null normal and denervated muscles under acetylcholine stimulation. MMP3 knockout muscles showed greater activation with acetylcholine after denervation than wild-type counterparts. *p<0.05. Graphical bars indicate standard error of the mean.

FIG. 9 depicts, in accordance with an embodiment herein, target organ reinnervation is more effective in matrix metalloproteinase 3 (MMP3) knockout animals. (A) Rise in compound motor action potential (CMAP) amplitude as measured from the tibialis anterior muscle is greater in MMP3 knockout mice compared to wild-type mice following nerve repair over a 10-week time period. **p<0.01 ***p<0.001. (B) Likewise, a greater proportion of endplates was innervated in MMP3 knockout mice than in wild-type mice at 4 and 10 weeks after nerve repair. A total of 50 endplates were evaluated per muscle. In some instances in wild-type specimens, endplate dispersion had occurred to such an extent that <50 endplates could be sampled. Y-axis represents the percentage of receptors demonstrating evidence of reinnervation. AChR 5 acetylcholine receptor. (C-E) Representative images of an endplate spared from denervation injury, a wild-type endplate 10 weeks after nerve repair, and an MMP3 knockout endplate 10 weeks after nerve repair. Note multiple points of nerve terminal-endplate contact denoted by arrowheads in E3 and absence of contact in D3. BTX 5 a-bungarotoxin; NF/Syn 5 neurofilament and synaptophysin. Original magnification, 3100; scale bars 5 1 lm. (F) Cross-sectional area analysis of the extensor digitorum longus (EDL) muscle after 10 weeks of nerve repair revealed larger mean fiber diameter in MMP3 knockout mice than in wild-type counterparts. (G-I) Representative images of muscle cross sections of the EDL in uninjured animals, and wild-type and MMP3 knockout animals 10 weeks after nerve repair. Original magnification, 340; scale bar 540 lm. *p<0.05. Graphical bars indicate standard error of the mean.

FIG. 10 depicts, in accordance with an embodiment herein, a schematic of agrin released by the nerve terminal into the muscle membrane. Acetylcholine receptors then aggregate to form the motor end plate. When the nerve is injured, the distal segment undergoes Wallerian degeneration. MMP-3 is an enzyme that degrades agrin, and then removes agrin from the muscle membrane, leading to motor endplate disassembly. In one embodiment, the present invention provides preservation of the motor end plate in an individual after traumatic nerve injury by agrin overexpression at the motor end plate via disruption of MMP 3 action.

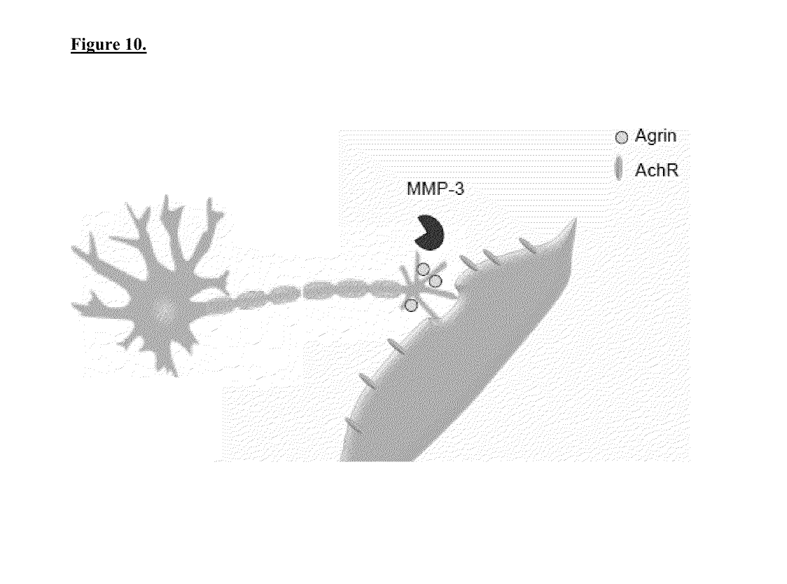

FIG. 11 depicts, in accordance with an embodiment herein, the finding that the wnt signaling pathway impacts the neuromuscular junction at the post synaptic level. (A) Wnt3a (green) can be seen localized to the acetylcholine receptors (AChR, red) and the motor nerve terminal (blue) in uninjured animals. The nerve terminal degenerated from the endplate at 1 month and 2 months post-transection and Wnt3a was upregulated at both timepoints. (B) and (C) depict band density as measured on western blots for 2 month gastroc-soleus complexes from uninjured and transected muscles for Wnt3a and beta-catenin, respectively.

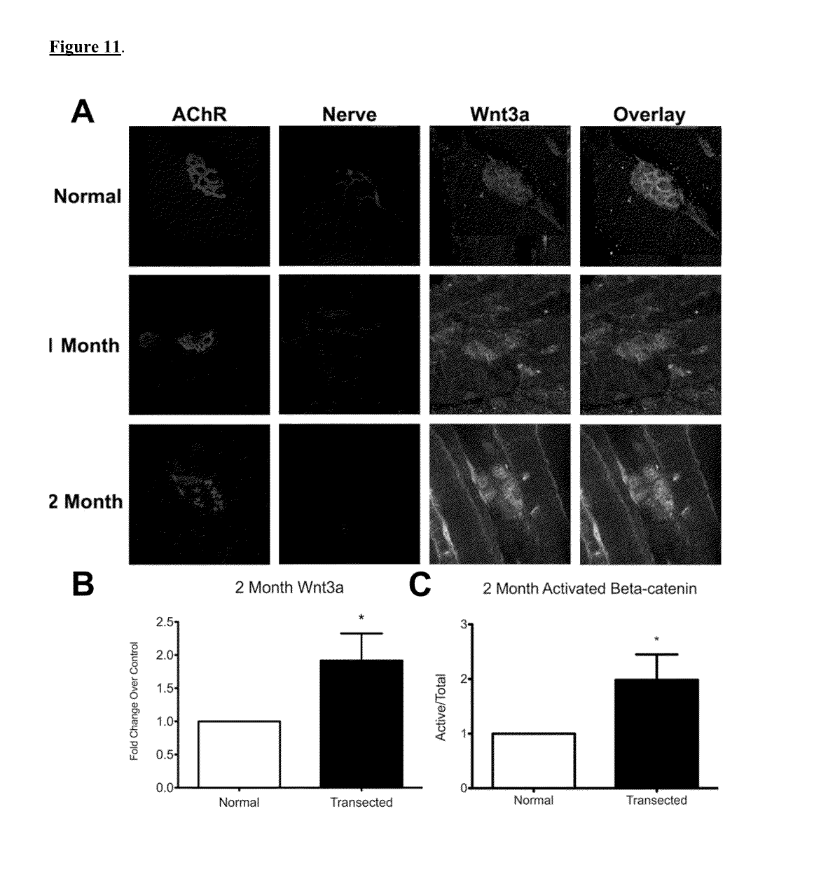

FIG. 12 depicts, in accordance with an embodiment herein, the finding that the wnt signaling pathway impacts the neuromuscular junction at the post synaptic level. Laser confocal image of the normal (D) and denervated (E) muscles of TCF/Lef:H2B-GFP mice shows nuclear-localized GFP fluorescence (green). The data suggests that the number of GFP positive cells was increased in the denervated muscles.

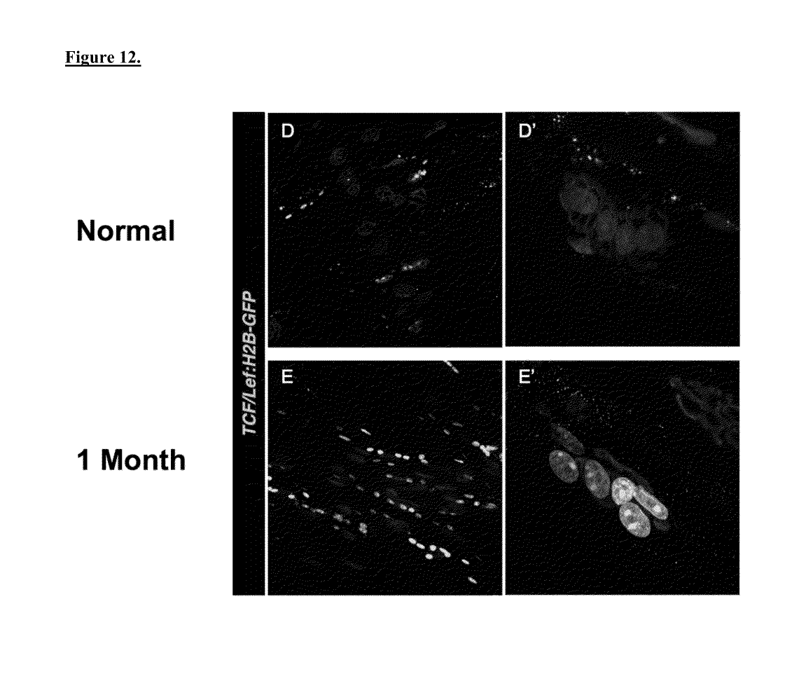

FIG. 13 depicts the Wnt signaling pathway. The Wnt signaling proteins play an important role in the development and the maintenance of the neuromuscular junction. Specifically, Wnt3a inhibits agrin-induced acetylcholine receptor clustering by suppressing rapsyn expression via beta-catenin dependent signaling.

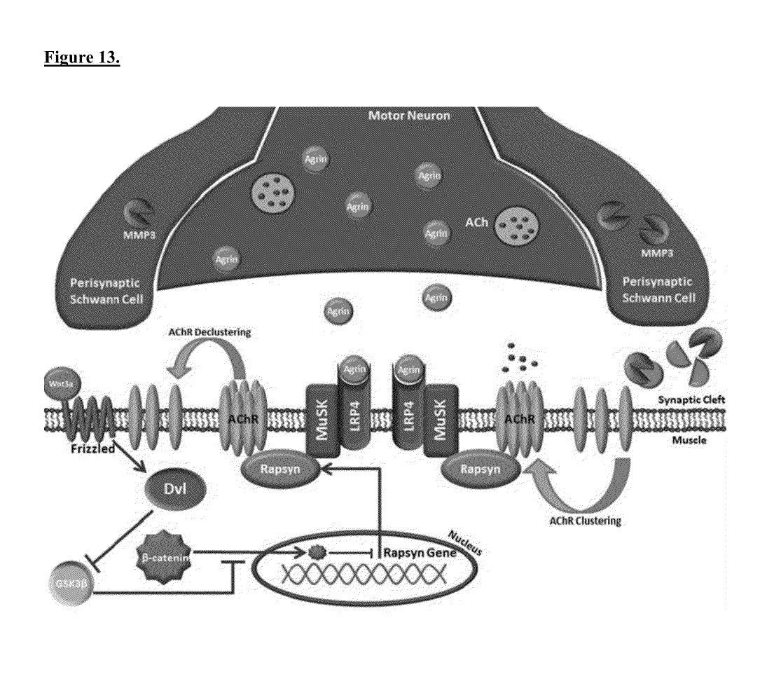

FIG. 14 depicts, in accordance with an embodiment herein, Wnt3a and beta-catenin are associated with NMJ destabilization following traumatic nerve injury. The inventor quantified levels of Wnt3a and activated beta-catenin in a mouse sciatic nerve transection model. Western blotting demonstrated that Wnt3a and beta-catenin protein levels were elevated at 2 months post-injury relative to controls. Immunohistochemistry of plantaris muscles demonstrated Wnt3a expression in the post-synaptic muscle, specifically at degrading AChR clusters.

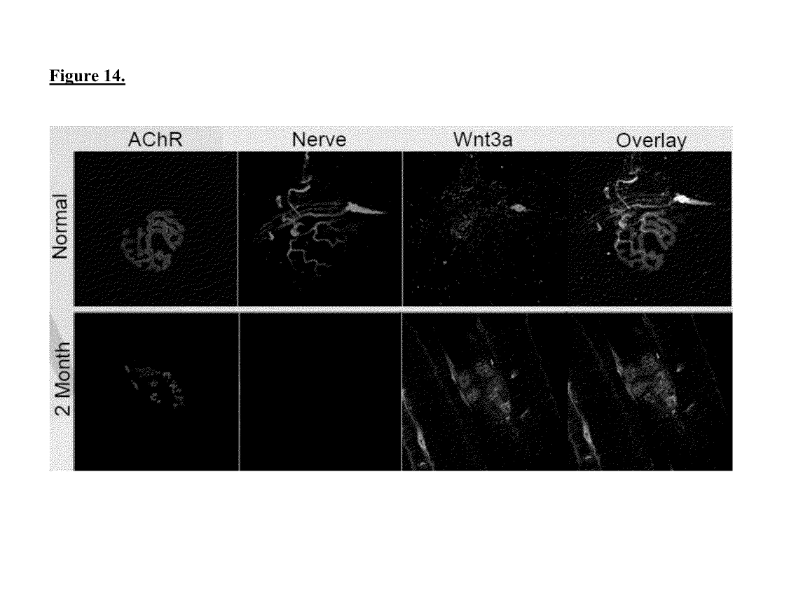

FIG. 15 depicts, in accordance with an embodiment herein, TCF/Lef:H2B-GFP Reporter Mice, where blue is DAPI, green is GFP, red is Alpha-Bungarotoxin, and purple is neurofilament. Transgenic mice that report Wnt/beta-catenin signaling activity were analyzed. The motor endplate of uninjured plantaris muscle with minimal GFP fluorescence. On the other hand, the number of GFP positive cells was increased in the denervated muscles at the acetylcholine receptor band. The data show that post-synaptic acetylcholine receptors at the NMJ destabilize after denervation by a process that involves the Wnt/beta-catenin pathway. As such, the Wnt/beta-catenin pathway is a useful therapeutic target to prevent the motor endplate degeneration that occurs following transection injuries.

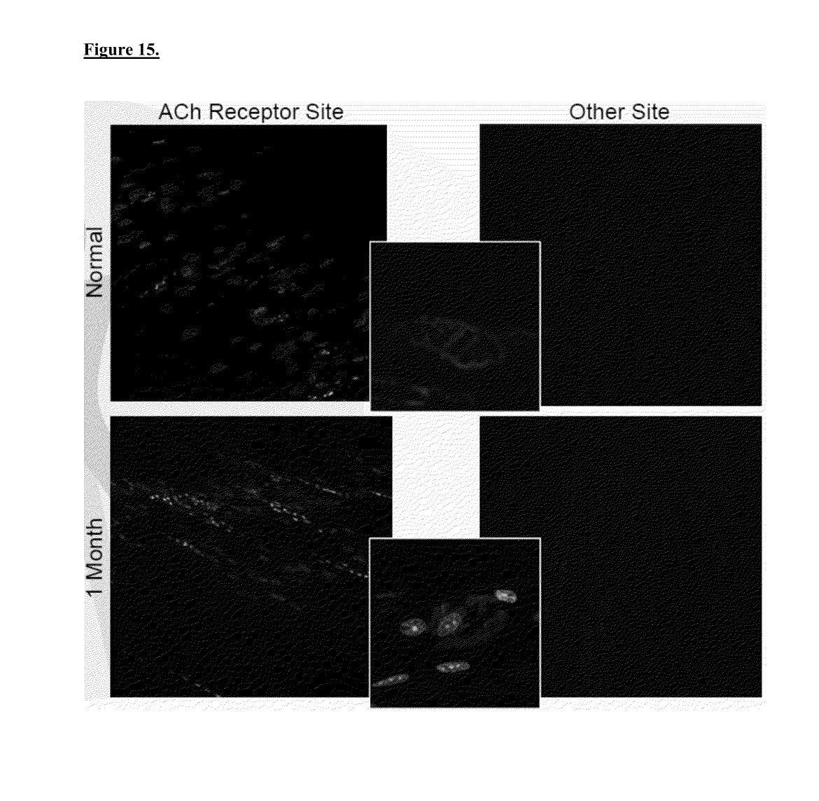

FIG. 16 depicts, in accordance with an embodiment herein, a chart of the number of GFP positive cells.

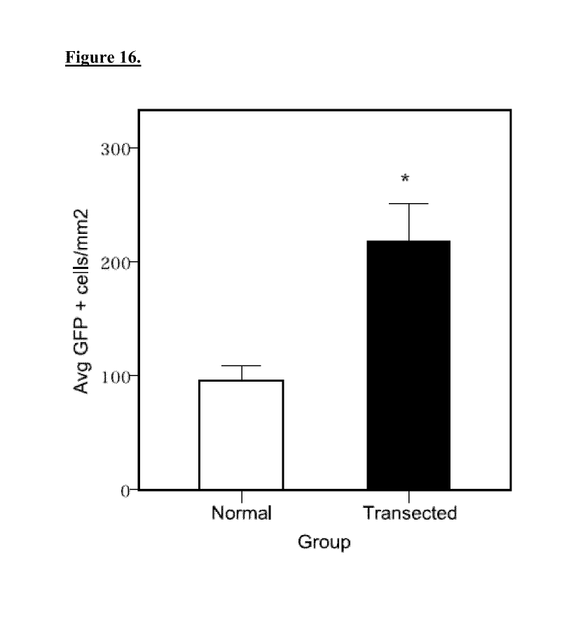

FIG. 17 depicts, in accordance with an embodiment herein, a chart of the expression H2B-GFP.

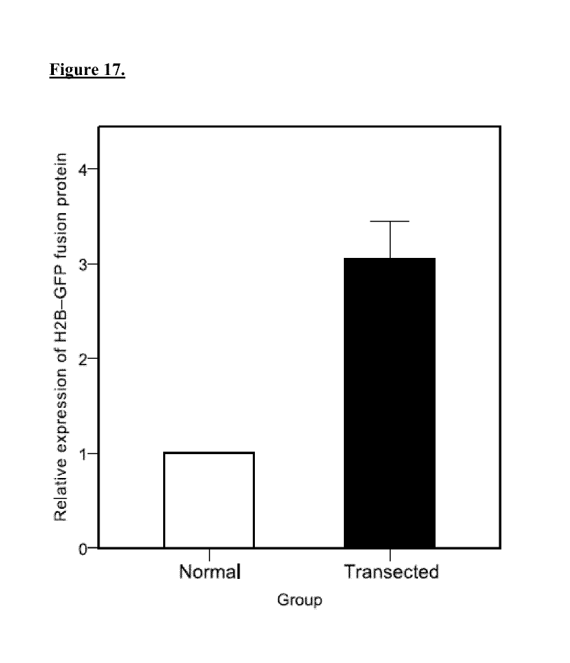

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As used herein, the term “MMP3” is an abbreviation for matrix metalloproteinase 3.

As used herein, the term “AChRs” is an abbreviation for acetylcholine receptors.

As used herein, the term “NMJ” is an abbreviation for neuromuscular junction.

As described herein, assembly of the motor endplate during early development depends on the interaction between agrin and its receptor muscle-specific kinase (MuSK). Agrin is synthesized at the neuromuscular junction by neurons and perisynaptic Schwann cells. During development, agrin triggers clustering of AChRs. Agrin levels are controlled in part through degradation by matrix metalloproteinase 3 (MMP3), which is secreted by perisynaptic Schwann cells. The inventor found that preservation of the motor end plate after traumatic nerve injury is possible by agrin overexpression at the motor end plate via disruption of MMP3 action.

As further disclosed herein, the inventor investigated the effect of preserving agrin on the stability of denervated endplates, and examined the changes in endplate structure following traumatic nerve injury in MMP3 knockout mice. After creation of a critical size nerve defect to preclude reinnervation, the inventor characterized the receptor area, receptor density, and endplate morphology in denervated plantaris muscles in wild-type and MMP3 null mice. The level of agrin and muscle-specific kinase (MuSK) was assessed at denervated endplates. In addition, denervated muscles were subjected to ex vivo stimulation with acetylcholine. Finally, reinnervation potential was compared after long-term denervation. The results were that in wild-type mice, the endplates demonstrated time-dependent decreases in area and receptor density and conversion to an immature receptor phenotype. In contrast, all denervation-induced changes were attenuated in MMP3 null mice, with endplates retaining their differentiated form. Agrin and MuSK were preserved in endplates from denervated MMP3 null animals. Furthermore, denervated muscles from MMP3 null mice demonstrated greater endplate efficacy and reinnervation. Thus, the results demonstrate a critical role for MMP3 in motor endplate remodeling, and reveal targets for therapeutic intervention to prevent motor endplate degradation following nerve injury.

In one embodiment, the present invention provides a method of treatment of nerve and/or muscle injury in an individual by administering a composition comprising an inhibitor of the MMP3 signaling pathway to the individual. In another embodiment, the inhibitor of the MMP3 signaling pathway is an inhibitor of MMP3. In another embodiment, the composition is administered to the individual by direct injection, intravenously and/or orally. In another embodiment, the composition is administered in conjunction with one or more surgical procedures and/or alternative treatments. In another embodiment, the composition is administered after a nerve injury and before surgical treatment. In another embodiment, the composition is administered after a nerve injury and after surgical treatment. In another embodiment, the muscles are denervated plantaris muscles. In another embodiment, the MMP3 inhibitor is an antibody. In another embodiment, the MMP3 inhibitor is a small molecule. In another embodiment, administering the composition results in motor endplate stability. In another embodiment, the individual is a human. In another embodiment, the individual is a rodent.

In one embodiment, the present invention provides a method of preserving the motor end plate after nerve injury in a subject, comprising providing a composition comprising MMP3 pathway specific siRNA, WNT pathway specific siRNA, and beta-catenin pathway specific siRNA, and transfecting one or more cells of the subject with the composition. As apparent to one of skill in the art, there are several methods readily available to provide siRNA sequences or transfection. Similarly, apparent to one of skill in the art, there are several genetic sequences that may be used to provide siRNA sequences. For example, as used herein, the MMP3 gene may be silenced by siRNA transfection MMP-3 Forward: 5-GTCTCTTTCACTCAGCCAAC-3 (SEQ. ID. NO.: 1) and Reverse: 5-ATCAGGATTTCTCCCCTCAG-3 (SEQ. ID. NO.: 2).

Similarly, as used herein, there are any number of MMP3 inhibitors that may be used in conjunction with various embodiments herein. Some examples of MMP3 inhibitors are the following compounds readily available to one of skill in the art: minocycline, MMP Inhibitor II, MMP Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618.

In one embodiment, the present invention provides a method of stabilizing a motor endplate in an individual by increasing agrin levels in the individual. In another embodiment, agrin levels are increased by inhibiting one or more molecules in the MMP3 signaling pathway in the individual. In another embodiment, the agrin levels are increased by inhibiting MMP3.

In one embodiment, the present invention provides a method of preventing nerve injury in an individual by administering a composition comprising an inhibitor of the MMP3 signaling pathway. In another embodiment, the inhibitor of the MMP3 signaling pathway is an MMP3 inhibitor. In another embodiment, administering the composition prevents motor endplate degradation in the individual.

In one embodiment, the present invention provides a composition comprising an MMP3 inhibitor and a pharmaceutically acceptable carrier.

In another embodiment, the present invention provides a method of treatment of nerve and/or muscle injury in an individual by administering a composition comprising agrin to the individual. In another embodiment, the composition is administered to the individual by direct injection, intravenously and/or orally. In another embodiment, the composition is administered in conjunction with one or more surgical procedures and/or alternative treatments. In another embodiment, the composition is administered after a nerve injury and before surgical treatment. In another embodiment, the composition is administered after a nerve injury and after surgical treatment. In another embodiment, administering the composition results in motor endplate stability. In another embodiment, the individual is a human. In another embodiment, the individual is a rodent.

In one embodiment, the present invention provides a method of preventing nerve injury in an individual by administering a composition comprising agrin. In another embodiment, administering the composition prevents motor endplate degradation in the individual.

In one embodiment, the present invention provides a composition comprising agrin and a pharmaceutically acceptable carrier.

As further disclosed herein, the inventors believed that Wnt signaling proteins (“Wnt signaling pathway”) also play an important role in the development and the maintenance of the neuromuscular junction (NMJ). Specifically, the inventors believed that Wnt3a and beta-catenin are associated with the NMJ destabilization following traumatic nerve injury. They quantified levels of Wnt3a and activated beta-catenin at various time-points in a murine nerve transection model to determine if NMJ destabilization is associated with increased concentration of these proteins within the motor endplate. A 10 mm segment of the right sciatic nerve was excised in both 129 SV/EV wildtype (WT) mice as well as in a transgenic mouse line expressing fluorescent reporter for WNT/beta-catenin signaling (TCF/Lef:H2B-GFP). The contralateral nerve of each animal was mobilized and served as an internal control. At 1 month and 2 months post injury, the gastrocsoleus and plantaris muscles were harvested, with Western blotting demonstrating that Wnt3a protein levels were elevated at 1 month (0.633±0.0540 vs 0.937±0.128) and 2 months post-injury (0.488±0.0170 0.970±0.232; p<0.002) relative to controls. Moreover, activated beta-catenin showed a similar increase (0.532±0.0250 vs. 1.050±0.204; p<0.026). Immunohistochemistry of WT muscles demonstrated that Wnt3a was up-regulated and recruited into the post-synaptic muscle, specifically to the degrading AChRs and motor endplate band at increasing levels until 2 months. Additionally, the data demonstrates that the number of GFP positive cells was increased in the denervated muscles of TCF/Lef:H2B-GFP mice. Taken together, post-synaptic AChRs at the NMJ appear to destabilize after denervation by a process that involves the Wnt/beta-catenin pathway. As such, the Wnt/beta-catenin pathway is a useful therapeutic target to prevent the motor endplate degeneration that occurs following transection injuries.

In one embodiment, the present invention provides a method of treatment of nerve and/or muscle injury in an individual by administering a composition comprising an inhibitor of the WNT and/or beta-catenin signaling pathway to the individual. In another embodiment, the inhibitor of the WNT and/or beta-catenin signaling pathway is an inhibitor of WNT3. In another embodiment, the inhibitor of the WNT and/or beta-catenin signaling pathway is an inhibitor of beta-catenin. In another embodiment, the composition is administered to the individual by direct injection, intravenously and/or orally. In another embodiment, the composition is administered in conjunction with one or more surgical procedures and/or alternative treatments. In another embodiment, the composition is administered after a nerve injury and before surgical treatment. In another embodiment, the composition is administered after a nerve injury and after surgical treatment. In another embodiment, the muscles are denervated plantaris muscles. In another embodiment, the WNT and/or beta-catenin signaling pathway inhibitor is an antibody. In another embodiment, the WNT and/or beta-catenin signaling pathway inhibitor is a small molecule. In another embodiment, administering the composition results in motor endplate stability. In another embodiment, the individual is a human. In another embodiment, the individual is a rodent.

As used herein, there are any number of inhibitors of WNT/beta-catenin signaling that may be used in conjunction with various embodiments herein. Some examples of small molecule inhibitors of WNT/beta-catenin signaling pathways are the following compounds readily available to one of skill in the art: XAV939, IWR1, IWP-1, IWP-2, JW74, JW55, okadaic acid, tautomycein, 2-[4-(4-fluoro-phenyl)piperazin-1-yl]-6-methylpyrimidin-4(3H)-one, niclosamide, cambinol, sulindac, filipin, bosutinib, imatinib, ethacrynic acid, PKF118-744, BC21, and Rp-8-Br-cAMP.

In one embodiment, the present invention provides a method of stabilizing a motor endplate in an individual by increasing agrin levels in the individual, wherein agrin levels are increased by inhibiting one or more molecules in the WNT and/or beta-catenin signaling pathway in the individual. In another embodiment, the agrin levels are increased by inhibiting Wnt3a. In another embodiment, the agrin levels are increased by inhibiting beta-catenin.

In one embodiment, the present invention provides a method of stabilizing a motor endplate in an individual by increasing AChR clustering levels in the individual, wherein AChR clustering levels are increased by inhibiting one or more molecules in the WNT and/or beta-catenin signaling pathway in the individual. In another embodiment, the AChR clustering levels are increased by inhibiting Wnt3a. In another embodiment, the AChR clustering levels are increased by inhibiting beta-catenin.

In one embodiment, the present invention provides a method of preventing nerve injury in an individual by administering a composition comprising an inhibitor of the WNT and/or beta-catenin signaling pathway. In another embodiment, the inhibitor of the WNT and/or beta-catenin signaling pathway is an Wnt3a inhibitor. In another embodiment, the inhibitor of the WNT and/or beta-catenin signaling pathway is an beta-catenin inhibitor. In another embodiment, administering the composition prevents motor endplate degradation in the individual.

In one embodiment, the present invention provides a composition comprising an WNT and/or beta-catenin signaling pathway inhibitor and a pharmaceutically acceptable carrier.

In one embodiment, the present invention provides a composition comprising a pharmaceutically acceptable carrier and one or more of the following: agrin, an inhibitor of the MMP3 signaling pathway, an inhibitor of the WNT signaling pathway, and an inhibitor of the beta-catenin pathway. In another embodiment, the inhibitor of the WNT signaling pathway is an inhibitor of Wnt3a. In another embodiment, the inhibitor of the MMP3 signaling pathway is an inhibitor of MMP3.

In another embodiment, the present invention provides a method of treating nerve injury in an individual by providing a composition comprising a pharmaceutically acceptable carrier and one or more of the following: agrin, an inhibitor of the MMP3 signaling pathway, an inhibitor of the WNT signaling pathway, and an inhibitor of the beta-catenin pathway; and administering a therapeutically effective dosage of the composition to the individual.

The present invention is also directed to a kit to treat nerve injury. The kit is an assemblage of materials or components, including at least one of the inventive compositions. Thus, in some embodiments the kit contains a composition including agrin, inhibitors of MMP3 signaling pathway, WNT signaling pathway and/or beta-catenin signaling pathway, as described above.

The exact nature of the components configured in the inventive kit depends on its intended purpose. For example, some embodiments are configured for the purpose of treating nerve injury. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to preserve the neuromuscular junction. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

As readily apparent to one of skill in the art, any number of compounds, small molecules, and/or antibodies may be used to inhibit expression of the MMP3, Wnt3a and beta-catenin molecules. Similarly, as readily apparent to one of skill in the art, MMP3, Wnt3a and beta-catenin are part of overall signaling pathways. Thus, in addition to a direct inhibition of MMP3, Wnt3a, and beta-catenin there are also other potential therapeutic targets along the respective pathway that may be available to increase agrin levels (including administration of agrin itself), stabilize motor endplates and/or improve outcomes following denervation injury.

### EXAMPLES

The following examples are provided to better illustrate the claimed invention and are not to be interpreted as limiting the scope of the invention. To the extent that specific materials are mentioned, it is merely for purposes of illustration and is not intended to limit the invention.

One skilled in the art may develop equivalent means or reactants without the exercise of inventive capacity and without departing from the scope of the invention.

### Example 1

### Overall

Traumatic peripheral nerve injuries often produce permanent functional deficits despite optimal surgical and medical management. One explanation for the impaired target organ reinnervation is degradation of motor endplates during prolonged denervation. As described herein, the inventor investigated the effect of preserving agrin on the stability of denervated endplates. The inventor examined the changes in endplate structure following traumatic nerve injury in MMP3 knockout mice. After creation of a critical size nerve defect to preclude reinnervation, the inventor characterized the receptor area, receptor density, and endplate morphology in denervated plantaris muscles in wild-type and MMP3 null mice. The level of agrin and muscle-specific kinase (MuSK) was assessed at denervated endplates. In addition, denervated muscles were subjected to ex vivo stimulation with acetylcholine. Finally, reinnervation potential was compared after long-term denervation. The results were that in wild-type mice, the endplates demonstrated time-dependent decreases in area and receptor density and conversion to an immature receptor phenotype. In contrast, all denervation-induced changes were attenuated in MMP3 null mice, with endplates retaining their differentiated form. Agrin and MuSK were preserved in endplates from denervated MMP3 null animals. Furthermore, denervated muscles from MMP3 null mice demonstrated greater endplate efficacy and reinnervation. Thus, the results demonstrate a critical role for MMP3 in motor endplate remodeling, and reveal targets for therapeutic intervention to prevent motor endplate degradation following nerve injury.

### Example 2

### In Vitro Assessment of AChR Clustering

C212 cells were purchased from ATCC (Manassas, Va.). Cells were expanded and differentiated into myotubes as previously described. Five days following differentiation, myotubes were then treated overnight with 0.11 g His-labeled rat recombinant agrin (R&D Systems, Minneapolis, Minn.) or 0.11 g rat recombinant agrin incubated with 2.51 g MMP3 active subunit (Millipore, Billerica, Mass.) for 72 hours. Western blot was performed to confirm cleavage of agrin. After treatment of myotubes with Alexa 555-conjugated a-bungarotoxin (a-BTX; Invitrogen, Carlsbad, Calif.; 1:1,000), samples were fixed according to standard procedures for immunohistochemistry. Ten random fields at 40 magnification were evaluated by a blinded observer for AChR clustering under fluorescent microscopy as previously described. An AChR cluster was defined as an aggregate of at least 4 lm2. Three samples from each treatment group were analyzed.

### Example 3

### Animal Model

All procedures involving living animals were approved by the institutional animal care and use committee of the University of California at Irvine. Homozygous pairs of the 129 Sv/Ev and MMP3 knockout mice were a gift from Dr W. Yong at the University of Calgary. Generation of the MMP3 knockout mice has been detailed previously. Genotyping was performed by Transnetyx (Cordova, Tenn.). Body weight and sciatic function index (SFI) were performed to identify any gross phenotypic or AQ1 motor differences.

### Example 4

### Surgery

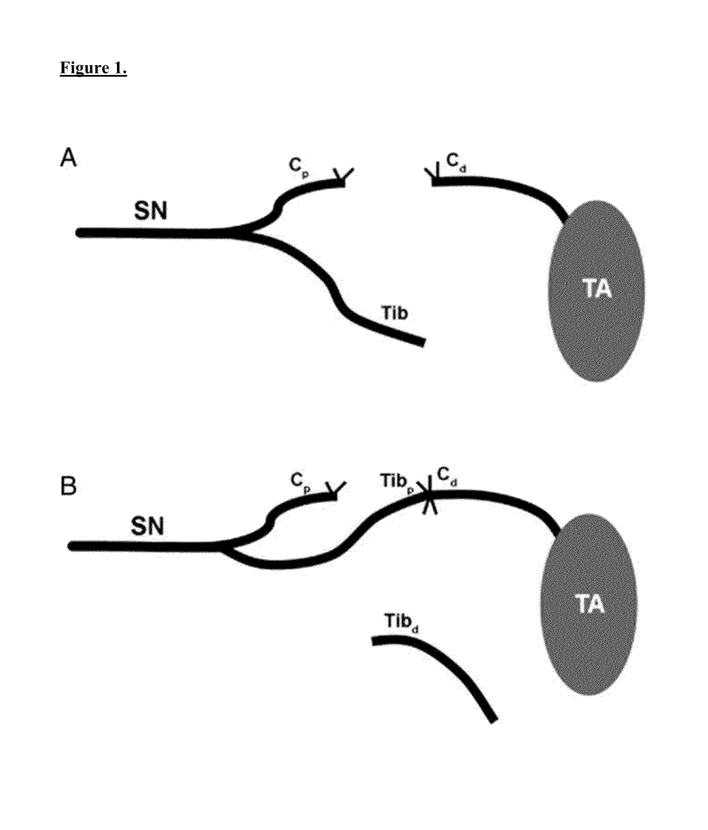

For denervation studies, 6-week-old male animals from either wild-type or MMP3 colonies were anesthetized with ketamine/xylazine. A 10 mm segment of the right sciatic nerve was excised. For regeneration studies, the tibialis anterior muscle was denervated for 2 months and subsequently reinnervated using a previously described technique (FIG. 1). Compound motor action potential (CMAP) recordings were performed biweekly by an experienced electrophysiologist blinded to the phenotype of the animals tested. M-waves were recorded from the tibialis anterior muscle. The reference electrode was inserted into the dorsal foot, and the stimulating electrode was inserted into ipsilateral lumbar paraspinal muscles.

### Example 5

### Immunohistochemistry

Whole mounts of plantaris muscles (n ¼ 4) were harvested ipsilateral and contralateral to transection injury in both wild-type and MMP3 knockout mice (for a list of antibodies, see Table 1). Following fixation, specimens were incubated in Alexa 555-conjugated a-BTX (Invitrogen; 1:1,000) and primary antibodies overnight. After rinsing, specimens were then incubated in Alexa 488 antimouse or Alexa 488 antirabbit (1:400). Visualization was performed under confocal microscopy. Evaluation of endplate area, pixel density, and morphology was conducted by a blinded observer.

TABLE 1
TABLE: List of Antibodies Used during the Study

| | Concen- | Appli- | |
|---|---|---|---|
| Antibody | tration | cation | Company |
| α-BTX (Alexa | 1:1,000 | IHC | Invitrogen, |
| 555) | | | Carlsbad, CA |
| Mouse monoclonal | 1:100 | IHC | Enzo Life Sciences, |
| antiagrin | | | Plymouth Meeting, |
| | | | PA |
| Mouse monoclonal | 1:50,000 | WB | Fitzgerald |
| anti-GAPDH | | | Industries, |
| | | | Action, MA |
| Mouse monoclonal | 1:100 (IP, IHC), | IHC, | Cell Applications, |
| anti-MuSK | 1:1,000 (WB) | IP, WB | San Diego, CA |
| Mouse monoclonal | 1:500 | IHC | Sigma-Aldrich, |
| anti-NF 70, 160, | | | St Louis, MO |
| 200 | | | |
| Mouse monoclonal | 1:1,000 | WB | Santa Cruz |
| antiphosphotyrosine | | | Biotechnology, |
| | | | Santa Cruz, CA |
| Mouse monoclonal | 1:500 | IHC | Covance, |
| SMI 312 | | | Emeryville, CA |
| Rabbit polyclonal | 1:1,000 | WB | Millipore, |
| anti-4G10 | | | Billerica, MA |
| Rabbit polyclonal | 1:1,000 | WB | Acris Antibodies, |
| anti-acetylcholine | | | Herford, Germany |
| receptor alpha | | | |
| Rabbit polyclonal | 1:100 | IP | Santa Cruz |
| anti-LRP4 | | | Biotechnology |
| Rabbit monoclonal | 1:200 (IHC), | WB and | Abcam, Cambridge, |
| anti-MMP3 | 1:1,000 (WB) | IHC | MA |
| Rabbit polyclonal | 1:500 | IHC | Dako, Carpinteria, |
| anti-5100 | | | CA |

All antibodies were diluted in blocking solution consisting of either 4% donkey serum/1% Triton-X in phosphate-buffered saline or 5% whole milk in tris-buffered saline with Tween.
α-BTX = α-bungarotoxin; GAPDH = glyceraldehyde-3-phosphate dehydrogenase; IHC = immunohistochemistry; IP = immunoprecipitaion; MMP3 = matrix metalloproteinase 3; MuSK = muscle-specific kinase; NF = neurofilament; WB = Western blot.

### Example 6

### Immunoblotting

Whole gastroc-soleus lysates were harvested from wild-type and MMP3 mice. Lysate protein concentration was determined using a BCA protein assay kit (Thermo Scientific, Rockford, Ill.). One hundred micrograms of protein was analyzed for all experiments. For evaluation of agrin and MuSK phosphorylation, immunoprecipitation was performed using antibodies to low-density lipoprotein receptor protein 4 (LRP4) or MuSK prior to blotting. Protein was then separated by 7.5% or 10% sodium dodecyl sulfate-polyacrylamide gel electrophoresis, transferred to nitrocellulose membranes, blocked with 5% dry skimmed milk, and incubated overnight at 4 C with primary antibodies. For detection, donkey antimouse secondary antibody conjugated with horseradish peroxidase (HRP; 1:10,000 dilution; Millipore) was used. Blots were developed with Western Chemiluminescent HRP Substrate (Thermo Scientific). Glyceraldehyde-3-phosphate dehydrogenase served as internal control when appropriate.

### Example 7

### Muscle Cross-Sectional Area

Plantaris muscles (n ¼ 4) from both ipsilateral and contralateral to the side of transection injury were cryoprotected in Tissue-Tek (Torrance, Calif.) OCT mounting medium. Twenty-micrometer sections were stained with hematoxylin and eosin. One hundred fifty fibers per muscle were then analyzed for cross-sectional area using ImageJ (NIH, Bethesda, Md.) software.

### Example 8

### Ex Vivo Stimulation

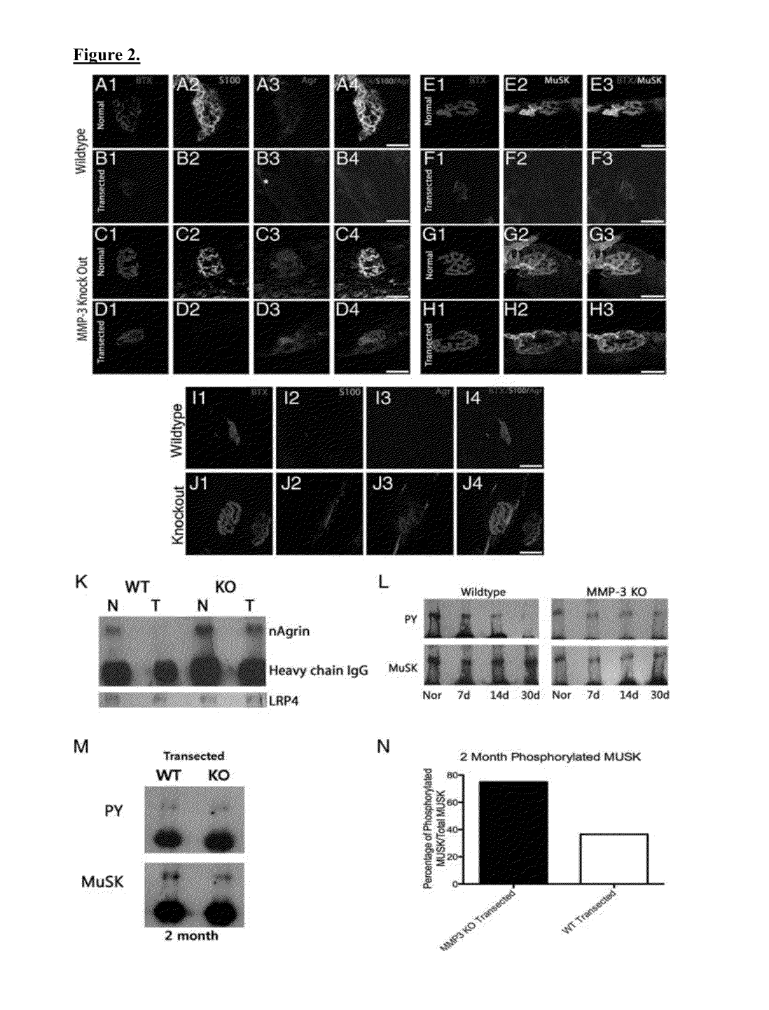

To assess muscle responses to acetylcholine, plantaris muscles were harvested from wild-type and MMP3 knockout mice 1 month postinjury (n ¼ 6 per group). Muscle length and mass were measured to ensure that these remained equal (see FIG. 2A, F2 B). The muscle was then mounted isometrically to a force transducer in a closed chamber with circulating O2 and mammalian Ringer solution. One molar acetylcholine was added to the chamber, and the maximum force was recorded over 10 minutes.

### Example 9

### Statistical Analysis

Data are presented as mean 6 standard error of the mean. One-way analysis of variance with Bonferroni post hoc comparison was performed unless otherwise indicated. Statistical significance is reported as p<0.05.

### Example 10

### MMP3 Deactivates the AChR Clustering of Agrin In Vitro

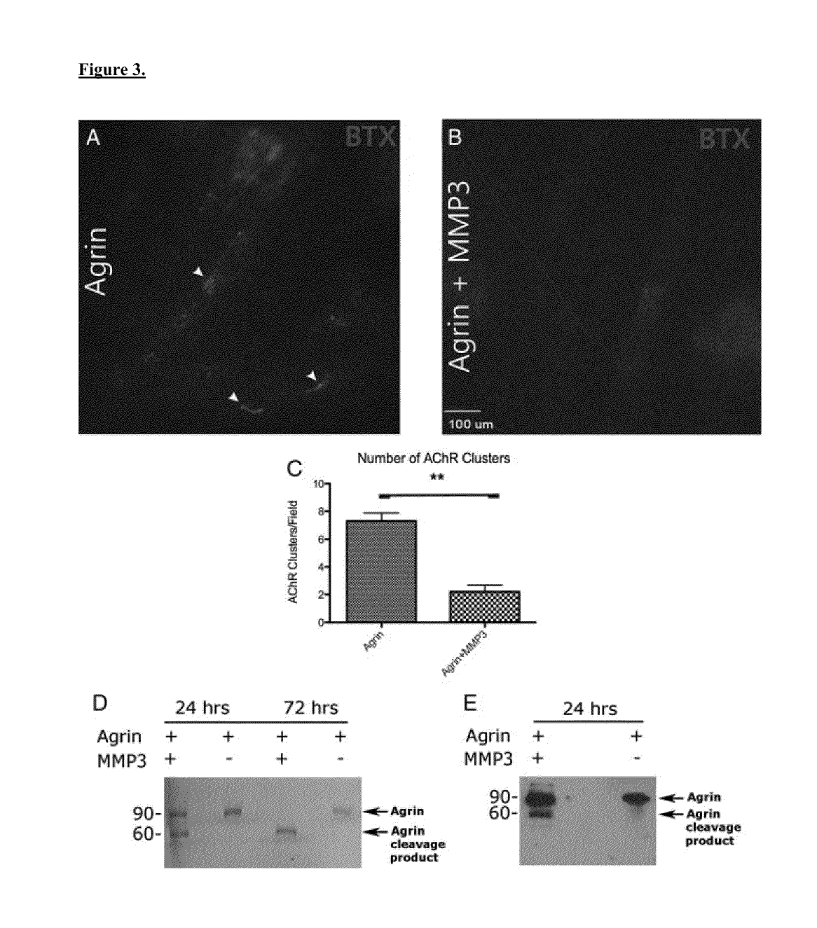

Recombinant agrin measuring approximately 90 kDa has been shown to induce aggregation of AChRs in C212 myotubes. To demonstrate that MMP3 inhibits the ability of agrin to induce clusters, we compared the clustering activity in vitro of recombinant rat agrin alone and recombinant rat agrin treated with MMP3. Multiple clusters were observed in myotubes treated solely with AQ3 agrin but not in cultures treated with agrin previously F3 incubated with MMP3 (FIG. 3). Quantification of clusters revealed that cultures treated with agrin exhibited a greater clustering ability than cultures treated with agrin processed by MMP3 (7.30 6 0.578 clusters/field vs 2.20 6 0.467 clusters/field). Agrin was cleaved by MMP3, resulting in a 60 kDa fragment as detected by silver stain. Treatment with MMP3 for 72 hours led to complete degradation of 90 kDa agrin. Probing with His antibody (1:1,000; Cell Signaling Technology, Danvers, Mass.) detected this 60 kDa fragment, indicating that MMP3 cuts agrin at the C-terminal site.

### Example 11

### MMP3 Deletion does not Affect Neuromuscular Development or Gross Motor Function

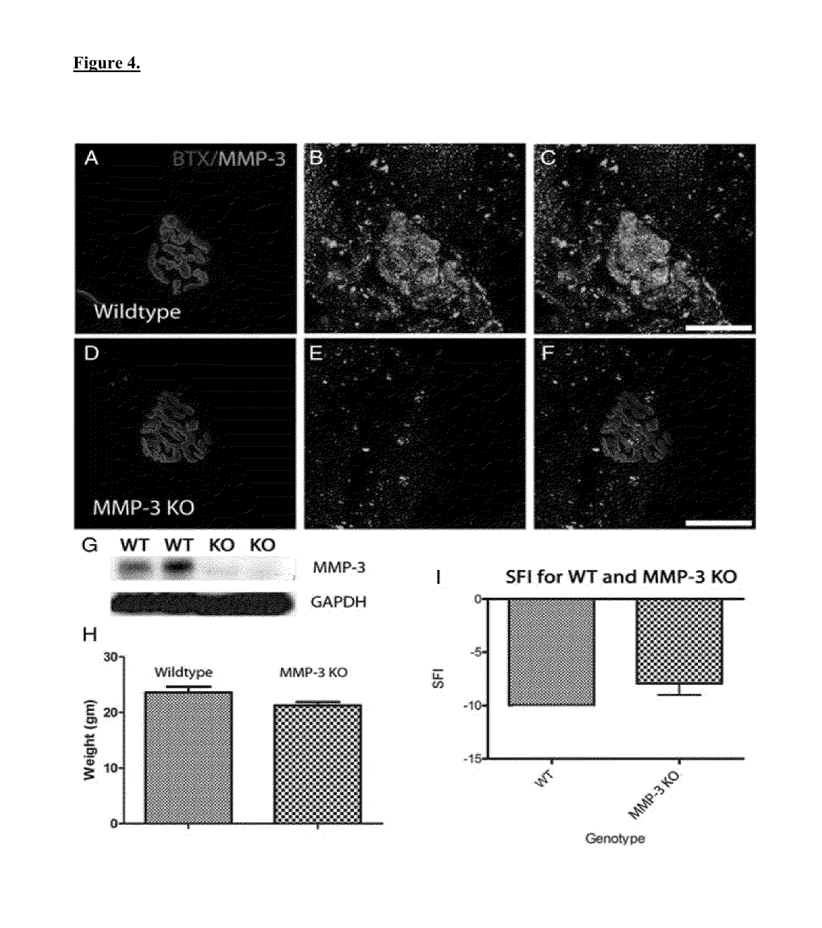

The inventor performed immunohistochemistry and Western blot for MMP3 protein to confirm deletion of MMP3 in knockout mice. MMP3 was undetectable at endplates or in muscle lysates in MMP3 null mice when compared to wild-type mice (FIG. 4A-F, G). The inventor found that wild-type and MMP3 knockouts had identical body weights at 6 weeks of age (wild type: 23.6 6 2.61 g vs MMP3 knockout: 21.3 6 1.42 g; see FIG. 4H). Furthermore, MMP3 knockout animals exhibited no motor deficiencies, as revealed by SFI analysis, which was within normal range (9.974 6 1.244, with p ¼ 0.480; see FIG. 4I).

### Example 12

### Remodeling of Motor Endplates after Denervation

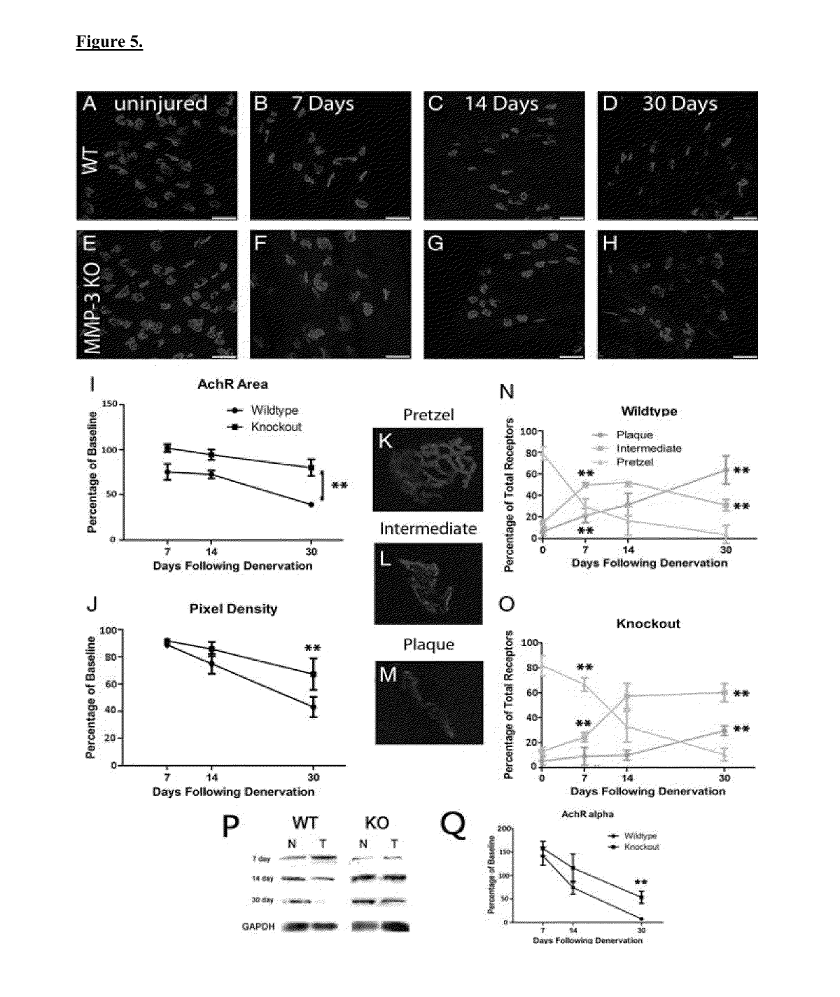

Deletion of MMP3 leads to formation of endplates with thicker junctional folds. The inventor questioned whether it might also protect against denervation-related degradation of motor endplates. Endplates from wild-type animals at several time points following denervation underwent progressive decreases in area and pixel density (area: 75.3 6 8.92% [1 week], 72.5 6 4.41% [2 weeks], 38.9 6 1.50% [1 month]; pixel density: 88.8 6 1.60% [1 week], 74.8 6 7.30% [2 weeks], 43.1 6 7.42% [1 month]; FIG. 5A-D, I, J). Surprisingly, the decline in F5 these 2 parameters was less significant in MMP3 null mice across the same time interval (area: 102 6 4.41% [1 week], 94.5 6 5.75% [2 weeks], 80.1 6 9.21% [1 month]; pixel density: 91.7 6 1.60% [1 week], 85.7 6 5.18% [2 weeks], 74.2 6 11.5% [1 month]; see FIGS. 5I, J). The overall difference between the endplate area and pixel density between wild-type and MMP3 groups was significant (p<0.01). A post hoc Bonferroni correction confirmed that the difference at all time points was significant in regard to endplate area, whereas differences in pixel density were significant at the 2-week and 1-month time points. There were also obvious changes in endplate morphology following denervation. In uninjured wild-type and MMP3 mice, normal endplates exhibited a weblike pattern with numerous perforations and septations (see FIG. 5). Following denervation, perforations and septations were diminished. To quantify this change, the inventor used a previously described scheme to characterize endplate AQ4 morphology. Endplates were categorized as pretzel (mature with weblike pattern including multiple perforations), plaque (immature and smaller size lacking perforations), and intermediate (morphology between that of plaque and pretzel). Most normal endplates in both wild-type and knockout mice exhibit a pretzel morphology. Plaque-type endplates increased up to 1 month denervation in wild-type mice (1 week: 29.4 6 9.21% pretzel vs 49.5 6 3.34% intermediate vs 21.1 6 8.41% plaque; 2 weeks: 16.4 6 9.24% pretzel vs 52.3 6 2.40% intermediate vs 31.4 6 9.31% plaque; 1 month: 3.81 6 9.42% pretzel vs 30.7 6 4.52% intermediate vs 63.6 6 10.4% plaque). In contrast, the intermediate phenotype was predominant at the 1-month time point in MMP3 null animals (1 week: 66.7 6 5.42% pretzel vs 24.3 6 3.54% intermediate vs 9.00 6 9.21% plaque; 2 weeks: 32.8 6 9.41% pretzel vs 57.3 6 5.34% intermediate vs 9.93 6 2.32% plaque; 1 month: 10.3 6 4.64% pretzel vs 60.2 6 3.24% intermediate vs 29.5 6 2.50% plaque). This conversion from a mature to a more immature endplate phenotype between the wildtype and MMP3 null mice was statistically significant throughout all injury time points (p<0.001). Bonferroni post hoc comparison revealed that the difference in intermediate and plaque-type receptors was significant at 1 week and 1 month.

### Example 13

### Denervation-Induced Changes in AChR Subunit a are Delayed in MMP3 Null Mice

To determine the concentration of receptors remaining at the endplate following denervation, the inventor quantified the amount of AChR a subunit by Western blot. Levels of AChR subunit a were elevated above baseline in both wild-type and MMP3 null mice at 1 week postdenervation (141.4 6 19.3% vs 157.5 6 15.2%; see FIGS. 5P, Q). By 2 weeks and 1 month, a subunit levels decreased drastically to 74.2 6 13.6% and 7.78 6 1.55% of control in wild-type mice. In contrast, a subunit levels were higher in MMP3 null mice at the 2-week and 1-month time points (116.0 6 29.9% and 53.5 6 12.9% of control). The overall difference in AChR a subunit concentration between wild-type and MMP3 knockout animals after denervation was significant (p<0.01); however, post hoc Bonferroni comparison revealed that only the difference at 1 month was significant.

### Example 14

### Endplates are Maintained in a Normal Topographic Distribution in MMP3 Null Mice after Prolonged Denervation

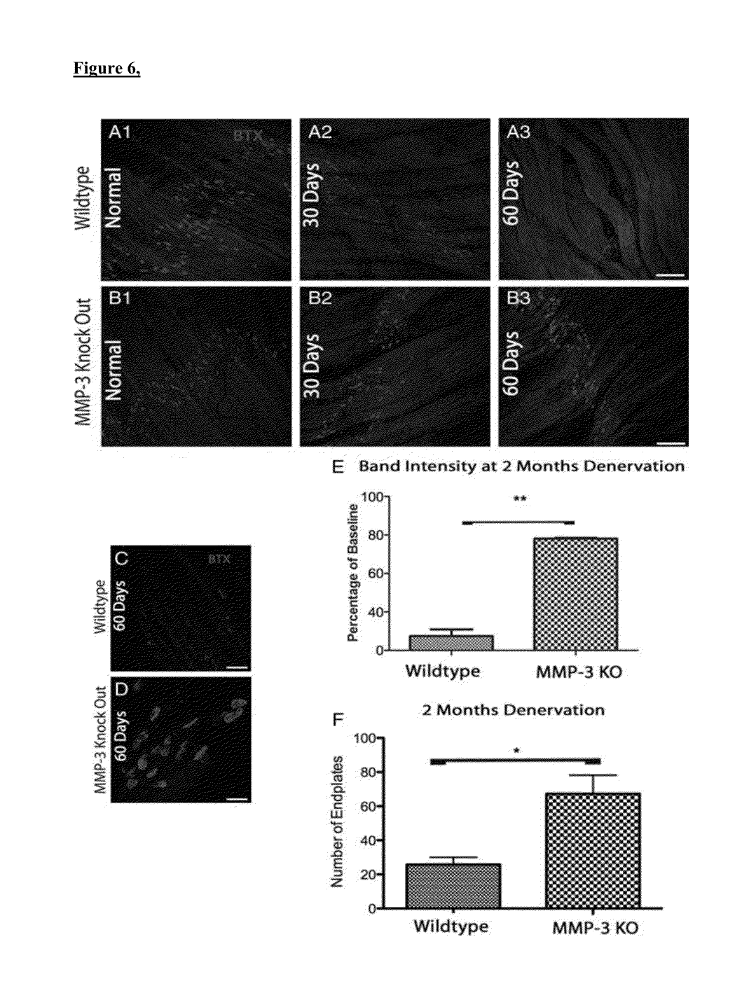

To determine whether MMP3 deletion slows endplate dispersion, we characterized the integrity of the endplate band. In normal muscle, AChR-rich endplates are distributed in a discrete band transversely across the muscle F6 substance (FIG. 6). Following denervation, the endplate band was still evident up to the 1-month time point in muscles from both mice but was absent following 2 months of denervation in wild-type mice. Surprisingly, the endplate band appeared relatively intact in MMP3 null mice despite 2 months of denervation (compare FIGS. 6A3 and B3). Higher-magnification images confirmed endplate dispersion in wild-type mice, whereas numerous pretzel-like endplates were still evident in MMP3 null mice. Band intensity measurements demonstrated that endplates from MMP3 null mice had a greater optical density value compared to wild-type mice (58.1+/−0.487% vs 7.06 6 3.58%, p<0.01). Likewise, the number of endplate counts at 2 months of denervation showed a significant decrease in the number of endplates in wild-type animals compared to MMP3 null mice (25.75 6 4.25 vs 67.25 6 10.93, p<0.05).

### Example 15

### Wallerian Degeneration Proceeds Normally in MMP3 Null Mice

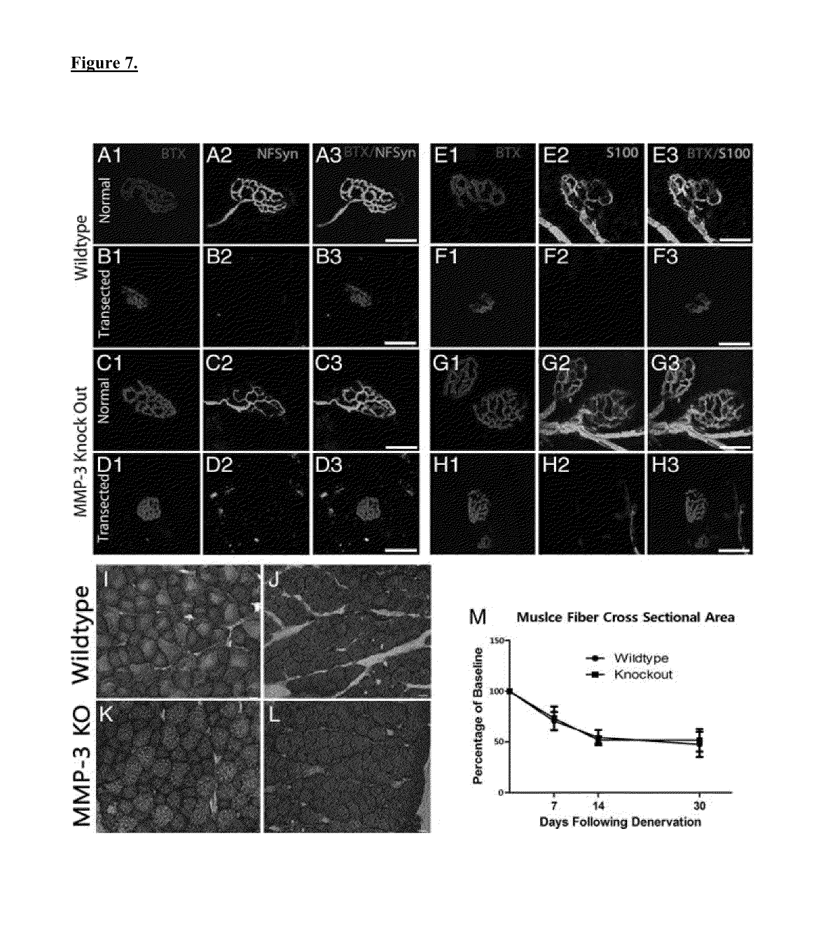

As delayed Wallerian degeneration has been shown to protect against neuromuscular destabilization, the inventor examined whether endplate stabilization in MMP3 null mice might be secondary to delayed Wallerian degeneration. In uninjured wild-type and MMP3 knockout muscles, neuromuscular contact was revealed by neurofilament and synaptophysin-positive endplates (FIG. 7). Following 1 and 2 F7 weeks of denervation, neural elements progressively retracted from the endplate. By 30 days post-transection, nerve terminals were undetectable in either wild-type or MMP3 null mice. These identical presynaptic patterns were evident despite strikingly different postsynaptic changes as characterized above. Similarly, double immunostaining for BTX and S100, a Schwann cell marker, revealed that in both wild-type and MMP3 null mice, S100 immunostaining was present at the endplate prior to injury. Following 30 days of denervation, S100 immunostaining was completely absent in both wild-type and MMP3 null mice. Thus, preservation of the endplate band in MMP3 null mice is not due to delayed Wallerian degeneration.

### Example 16

### Rate of Muscle Atrophy is Unaltered Despite MMP3 Deletion

Because slower muscle degradation can lead to relative endplate preservation, we assessed whether deletion of MMP3 decreased the rate of muscle atrophy. Measurements of muscle cross-sectional area revealed that atrophy occurred at equal rates in both wild-type (see FIGS. 71, J) and MMP3 null mice (see FIGS. 7K, L) following denervation injury (quantified in FIG. 7M). Thus, following denervation, muscle undergoes atrophy likely secondary to disuse; however, deletion of MMP3 has no effect on the rate of muscle atrophy.

### Example 17

### Deletion of MMP3 Preserves Agrin and MuSK at Denervated Motor Endplates

The inventor then determined whether deletion of MMP3 preserves agrin and downstream mechanisms at the motor endplate following long-term denervation. In wild-type mice, immunostaining for agrin and MuSK revealed that both were localized to the area of the primary gutters in endplates as previously documented (FIG. 2). Furthermore, agrin appeared to localize to perisynaptic Schwann cell terminals. In wild-type animals, agrin and MuSK immunofluorescence progressively declined during the 1-week and 2-week time points (not shown). By 1 month, there was minimal immunostaining for agrin or MuSK at the endplate. Conversely, both agrin and MuSK were present at denervated endplates in MMP3 null mice up to 2 months following denervation (compare FIG. 2C1-4, D3, D4, G1-3, H1-3, 11-4, J1-4). This result demonstrates that deletion of MMP3 prevents the removal of agrin and MuSK from the endplate region.

### Example 18

### MMP3 Deletion Preserves Neural Agrin Leading to Persistent MuSK Activity in Denervated Endplates

As no antibody currently exists to specifically detect neural agrin, the isoform responsible for endplate organization, the inventor coimmunoprecipitated muscle lysates with LRP4 antibody, which has a high affinity for neural but not muscle agrin. Upon reprobing muscle immunoprecipitates with agrin antibody, a 95 kD band was obtained (see FIG. 2K), a size consistent with the isoform necessary for AChR clustering. Furthermore, there was a substantial decrease in neural agrin in the wild-type but not MMP3 null mice in 1-month denervated samples (see FIG. 2K). These results indicate that MMP3 deletion preserves neural agrin at endplates following denervation. To assess the downstream effect of neural agrin preservation, the inventor characterized the extent of MusK phosphorylation. Immunoprecipitates obtained using MuSK antibody and probed with phosphotyrosine antibodies revealed a 110 kDa band representing phosphorylated MuSK (see FIG. 2L). The band intensity decreased incrementally in respect to total MuSK following 1 week, 2 weeks, 1 month, and 2 months of denervation in wildtype mice (see FIG. 2L-N). In contrast, MuSK remained heavily phosphorylated in MMP3 null mice even at 2 months of denervation. Thus, downstream mechanisms responsible for motor endplate maintenance remain functional in MMP3 null mice likely due to persistence of agrin.

### Example 19

### MMP3 Null Mice Demonstrate Retained Motor Endplate Efficacy Following Denervation

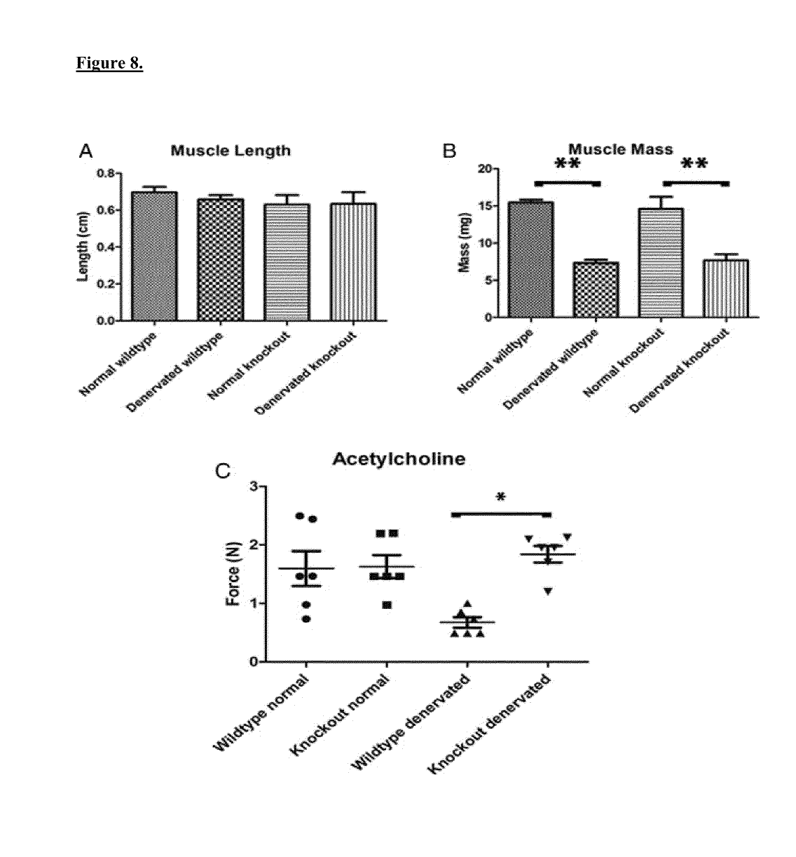

To assess endplate efficacy after denervation, the inventor measured muscle contractile force to externally applied acetylcholine in uninjured and 1-month denervated muscle. The contractile force in response to 1M acetylcholine was similar in uninjured muscles from wild-type and MMP3 null mice (1.60 6 0.733N and 1.63 6 0.481N; F8 FIG. 8C). However, the mean force was higher in denervated muscles from MMP3 null mice compared to wildtype (1.84 6 0.346N vs 0.674 6 0.221N; p<0.05). The higher generated force indicates that endplates were more functional in denervated MMP3 knockout mice.

### Example 20

### Knockout of MMP3 Improves Functional Nerve Regeneration

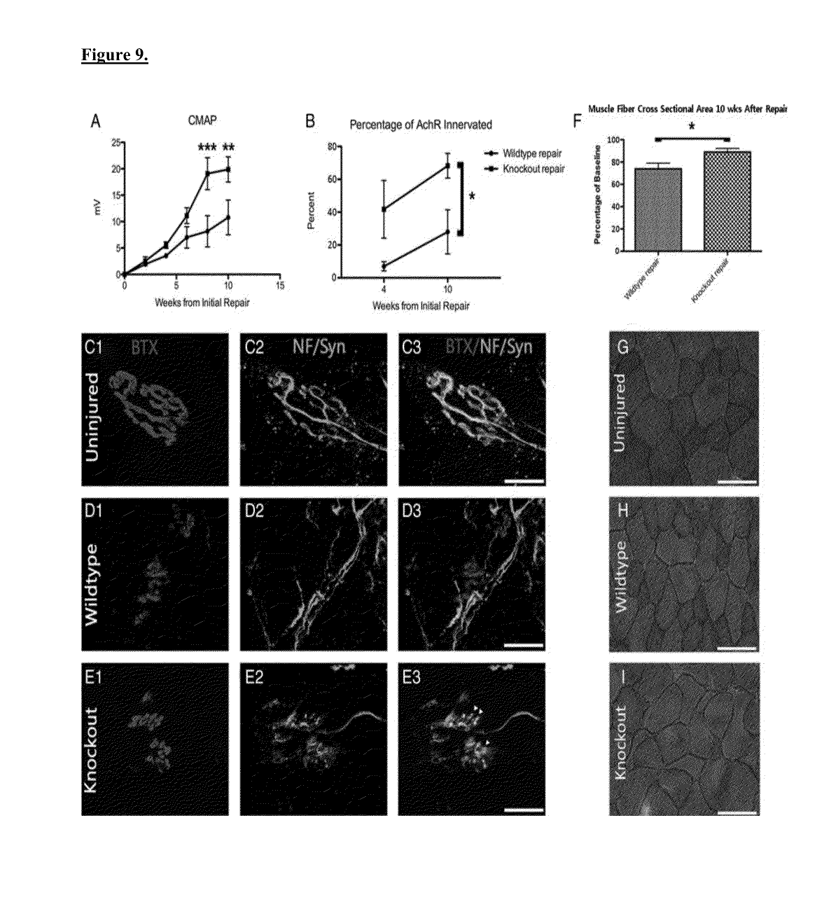

To determine whether preservation of motor endplate function in MMP3 null mice might improve functional recovery, they surgically reinnervated 2-month denervated tibialis anterior muscles in both wild-type and MMP3 null animals. Using a cross-suture paradigm, they transferred the proximal posterior tibial nerve to the distal stump of the common peroneal nerve after 2 months of denervation (see FIG. 1). Nerve regeneration was assessed serially using electrophysiology. Normal CMAP amplitudes of the uninjured side were calculated to be approximately 46.9 6 7.87 mV for the wild-type and 49.9 6 7.20 mV for the MMP3 null animals. CMAP recordings demonstrated progressive increases in amplitude in both wild-type and MMP3 null mice (FIG. 9A). By the 8- and F9 10-week time points, the amplitude in MMP3 null mice had risen substantially relative to wild-type (19.07 6 3.67 mV vs 8.17 6 4.02 mV for 8 weeks, p<0.001; and 19.9 6 2.52 mV vs 10.9 6 3.54 mV for 10 weeks, p<0.01). They also characterized endplates in the tibialis anterior at 4 and 10 weeks after nerve repair. A greater number of endplates were reinnervated in MMP3 null mice compared to wild-type (41.8 6 18.6% vs 7.09 6 5.60% for 4 weeks and 68.7 6 9.21% vs 28.4 6 11.4% for 10 weeks; see FIG. 9B; p<0.05). Advancing nerve terminals often failed to contact the endplate in wild-type mice but frequently entered the postsynaptic area in MMP3 knockout mice (see FIG. 9C-E). Likewise, the muscle cross-sectional area of the extensor digitorum longus at 10 weeks after nerve repair was larger in MMP3 null mice than in wild-types (88.0 6 3.21% vs 73.8 6 5.20%; see FIGS. 9F-I; p<0.05). Thus, outcomes following nerve repair appeared to be more robust in MMP3 null mice.

### Example 21

### MMP3 Generally

The inventor showed that genetic deletion of MMP3, which normally degrades agrin, leads to sustained agrin levels at denervated endplates, preserved phosphorylation of MuSK, and preservation of denervated endplates for at least 2 months following nerve degeneration. Here, the inventor has shown that neural agrin was depleted in wild-type denervated muscles but not in MMP3 knockout muscles. The presence of neural agrin in denervated MMP3 knockout muscles corresponded to greater downstream phosphorylation of MuSK. These data, combined with the observations on endplate morphology after denervation, link agrin persistence with enhanced stability of AChRs at the motor endplate. Long-term denervation of the tibialis anterior muscle resulted in significant compromise in electrodiagnostic outcomes following nerve repair. Although these results were considered to be due to degenerative mechanisms within the former neuromuscular interface, this idea was not investigated histologically. The inventor found that long-term denervation leads to profound atrophy in endplate structure, which translates to deficits in functional activation. Furthermore, these deficits were delayed in MMP3 knockout mice, thereby suggesting that preservation of endplate architecture can substantially improve functionality. The inventor presents evidence that neural repair following long-term denervation leads to improved functional endpoints when motor endplate stability is preserved secondary to MMP3 inactivation. The data identifies therapeutic targets to enhance outcomes during nerve regeneration.

### Example 22

### WNT3a and Beta-Catenin Signaling

Wnt signaling proteins (“Wnt signaling pathway”) play an important role in the development and the maintenance of the neuromuscular junction (NMJ). Specifically, the inventors believed that Wnt3a and beta-catenin are associated with the NMJ destabilization following traumatic nerve injury. They quantified levels of Wnt3a and activated beta-catenin at various time-points in a murine nerve transection model to determine if NMJ destabilization is associated with increased concentration of these proteins within the motor endplate. A 10 mm segment of the right sciatic nerve was excised in both 129 SV/EV wildtype (WT) mice as well as in a transgenic mouse line expressing fluorescent reporter for WNT/beta-catenin signaling (TCF/Lef:H2B-GFP). The contralateral nerve of each animal was mobilized and served as an internal control. At 1 month and 2 months post injury, the gastrocsoleus and plantaris muscles were harvested, with Western blotting demonstrating that Wnt3a protein levels were elevated at 1 month (0.633±0.0540 vs 0.937±0.128) and 2 months post-injury (0.488±0.0170 0.970±0.232; p<0.002) relative to controls. Moreover, activated beta-catenin showed a similar increase (0.532±0.0250 vs. 1.050±0.204; p<0.026). Immunohistochemistry of WT muscles demonstrated that Wnt3a was up-regulated and recruited into the post-synaptic muscle, specifically to the degrading AChRs and motor endplate band at increasing levels until 2 months. Additionally, the data demonstrates that the number of GFP positive cells was increased in the denervated muscles of TCF/Lef:H2B-GFP mice. Taken together, post-synaptic AChRs at the NMJ appear to destabilize after denervation by a process that involves the Wnt/beta-catenin pathway. As such, the Wnt/beta-catenin pathway is a useful therapeutic target to prevent the motor endplate degeneration that occurs following transection injuries.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of treating nerve injury in an individual, comprising:
providing a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway; and
administering a therapeutically effective dosage of the composition to the individual.

2. The method of claim 1, wherein the composition is administered in conjunction with surgical treatment.

3. The method of claim 1, wherein the individual is a human.

4. The method of claim 1, wherein the inhibitor of the MMP3 signaling pathway is an inhibitor of MMP3.

5. The method of claim 1, wherein the inhibitor of the WNT signaling pathway is an inhibitor of Wnt3a.

6. The method of claim 1, wherein the nerve injury is treated by preserving the neuromuscular junction (NMJ).

7. The method of claim 1, wherein administering the composition prevents degradation of the motor end plate after prolonged denervation.

8. The method of claim 1, wherein the composition is administered prior to nerve injury surgery.

9. The method of claim 1, wherein the composition is administered post nerve injury surgery.

10. The method of claim 1, wherein the composition is administered intravenously.

11. The method of claim 1, wherein the inhibitor of the MMP3 signaling pathway is selected from the following: minocycline, MMP Inhibitor II, MMP Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618.

12. The method of claim 1, wherein the inhibitor of the MMP3 signaling pathway is an MMP3 siRNA molecule.

13. The method of claim 1, wherein the inhibitor of the WNT signaling pathway is an Wnt3a siRNA molecule.

14. The method of claim 1, wherein the inhibitor of the WNT signaling pathway is an inhibitor of the armadillo protein β-catenin.

15. The method of claim 1, wherein the inhibitor of the WNT signaling pathway is an inhibitor of one or more of the following: beta-catenin destruction complex, WNT/Beta-catenin signalsome, cadherin junctions, and hypoxi sensing system Hif-1alpha (hypoxia induced factor 1beta).

16. The method of claim 1, wherein the inhibitor of the WNT signaling pathway is one or more of the following: XAV939, IWR1, IWP-1, IWP-2, JW74, JW55, okadaic acid, tautomycein, 2-[4-(4-fluoro-phenyl)piperazin-1-yl]-6-methylpyrimidin-4(3H)-one, niclosamide, cambinol, sulindac, filipin, bosutinib, imatinib, ethacrynic acid, PKF118-744, BC21, and Rp-8-Br-cAMP.

17. A composition comprising:
a therapeutically effective dosage of a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway; and
a pharmaceutically acceptable carrier.

18. The composition of claim 17, wherein the inhibitor of the MMP3 signaling pathway is an inhibitor of MMP3.

19. The composition of claim 18, wherein the inhibitor of MMP3 is an MMP3 antibody.

20. The composition of claim 18, wherein the inhibitor of MMP3 is selected from the following: minocycline, MMP Inhibitor II, MMP Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618.

21. The composition of claim 17, wherein the inhibitor of the WNT signaling pathway is an inhibitor of Wnt3a.

22. The composition of claim 21, wherein the inhibitor of Wnt3a is an Wnt3a antibody.

23. The composition of claim 17, wherein the inhibitor of MMP3 signaling pathway is selected from the following: XAV939, IWR1, IWP-1, IWP-2, JW74, JW55, okadaic acid, tautomycein, 2-[4-(4-fluoro-phenyl)piperazin-1-yl]-6-methylpyrimidin-4(3H)-one, niclosamide, cambinol, sulindac, filipin, bosutinib, imatinib, ethacrynic acid, PKF118-744, BC21, and Rp-8-Br-cAMP.

24. A method of preventing nerve injury in an individual, comprising:
providing a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway; and
administering a therapeutically effective dosage of the composition to the individual prior to nerve injury.

25. The method of claim 24, wherein the composition is administered intravenously.

26. A method of preserving the motor end plate after nerve injury in a subject, comprising:
providing a composition comprising MMP3 pathway specific siRNA, WNT pathway specific siRNA, and beta-catenin pathway specific siRNA; and
transfecting one or more cells of the subject with the composition.

27. The method of claim 26, wherein the composition comprises SEQ. ID. NO.: 1 and SEQ. ID. NO.: 2.

28. The method of claim 26, wherein the subject is a human.

29. The method of claim 26, wherein the subject is a rodent.
